(19) United States
(12) Patent Application Publication
(10) Pub. No.: US 2025/0352917 A1
(43) Pub. Date: Nov. 20, 2025

(54) STUFFED TOY

(71) Applicant: BANDAI CO., LTD., Tokyo (JP)

(72) Inventor: Mana IGARASHI, Tokyo (JP)

(21) Appl. No.: 19/067,814

(22) Filed: Feb. 28, 2025

(30) Foreign Application Priority Data
May 15, 2024 (JP) 2024-079296

(51) Int. Cl. A63H 3/00; A61F 7/08; A63H 3/02

(52) U.S. Cl. CPC A63H 3/005; A61F 7/08; A63H 3/02

(57) ABSTRACT

A stuffed toy includes a body including a head and a trunk and further includes a holder located in the trunk. The holder is configured to hold a heat storage member.

### CROSS-REFERENCE TO RELATED APPLICATIONS

This application claims priority to Japanese Patent Application No. 2024-079296 filed in the Japan Patent Office on May 15, 2024, the entire contents of which are incorporated herein by reference.

### BACKGROUND OF THE INVENTION

### 1. Field of the Invention

The present invention relates to a stuffed toy.

### 2. Description of the Related Art

Japanese Unexamined Patent Application Publication No. 2005-137576 discloses a cover including a body and a storage from which an ice pack or the like is removable. The body has an outer shape resembling, for example, an animal or a vehicle.

As discussed in Japanese Unexamined Patent Application Publication No. 2005-137576, the cover is formed as a stuffed toy and is configured such that the ice pack extends from a head of the stuffed toy to a trunk thereof. Such a configuration allows the whole of the stuffed toy to provide a cooling effect but restricts motion of the head and the trunk. Thus, the stuffed toy exhibits less motion and lacks in interest.

### SUMMARY OF THE INVENTION

It is an object of the present invention to provide a stuffed toy that is highly practical and highly interesting.

A stuffed toy according to an embodiment of the present invention includes: a body including a head and a trunk; and a holder located in the trunk. The holder is configured to hold a heat storage member.

### BRIEF DESCRIPTION OF THE DRAWINGS

FIG. 1 is a plan view of the front of a stuffed toy according to an embodiment of the present invention and a heat storage member;

FIG. 2 is a side elevation view of the stuffed toy and the heat storage member in FIG. 1;

FIG. 3 is a sectional view of the stuffed toy taken along line III-III in FIG. 1 illustrating a side elevation of the heat storage member that is to be held and a side elevation of the heat storage member in a first holding state;

FIG. 4 is a perspective view of the stuffed toy in a first display state;

FIG. 5 is a sectional view of the stuffed toy taken along line V-V in FIG. 1 illustrating a side elevation of the heat storage member in a second holding state;

FIG. 6 is a perspective view of the stuffed toy in a second display state;

FIG. 7 is an enlarged sectional view of part VII in FIG. 3; and

FIG. 8 is an enlarged sectional view of part VIII in FIG. 3 subjected to an external force.

### DESCRIPTION OF THE PREFERRED EMBODIMENTS

A stuffed toy according to an embodiment of the present invention will be described below with reference to the drawings.

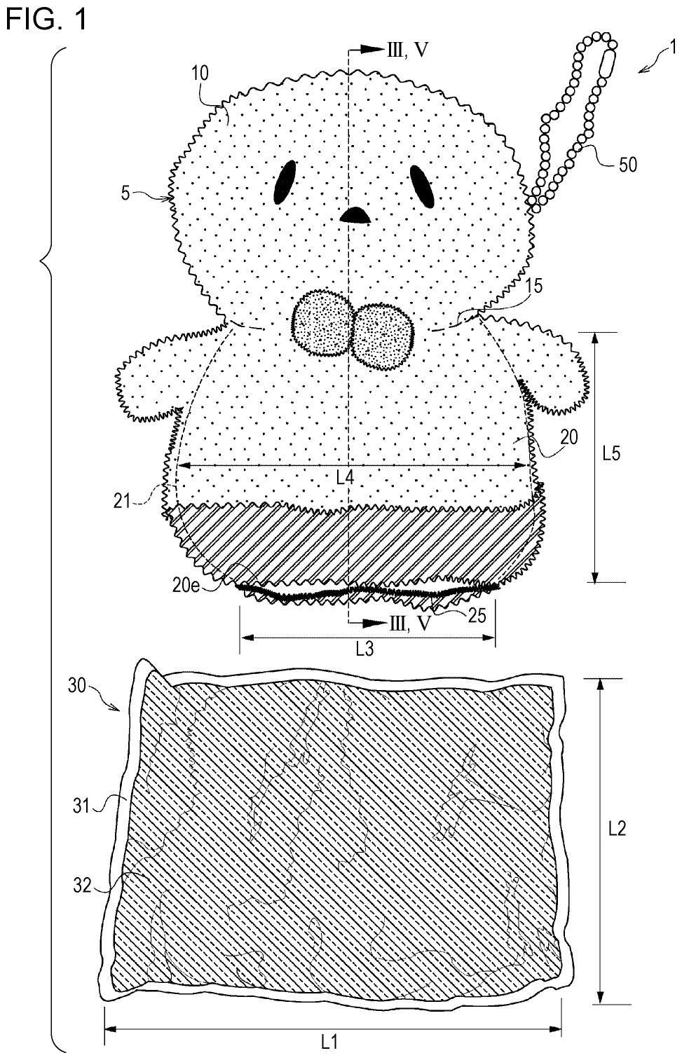

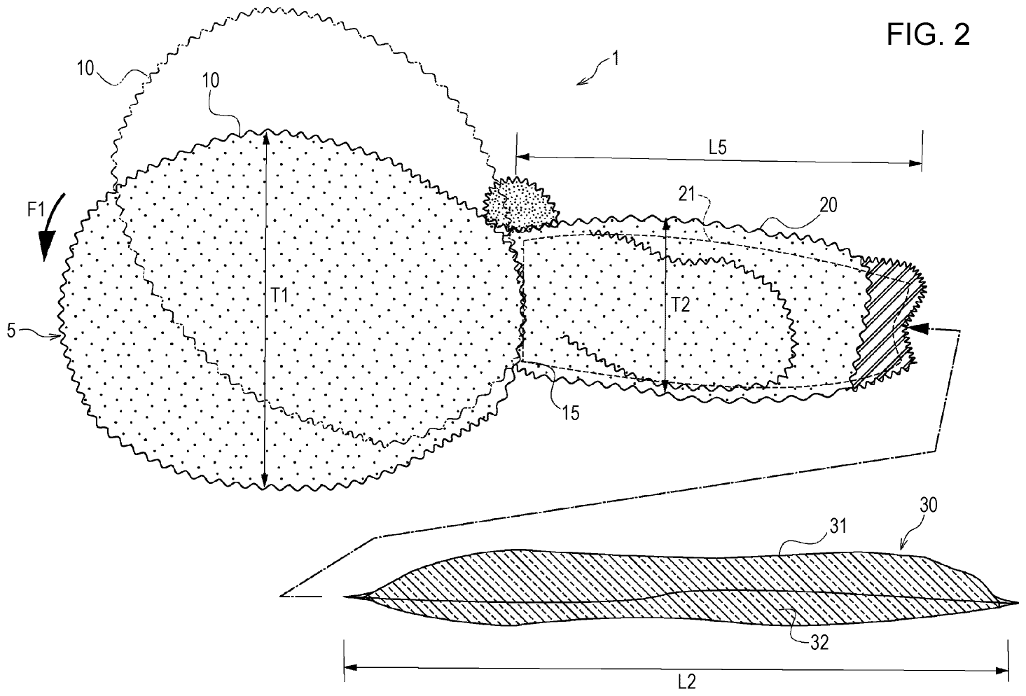

FIG. 1 is a plan view of the front of a stuffed toy 1 and a heat storage member 30. FIG. 2 is a side elevation view of the stuffed toy 1 and the heat storage member 30. Referring to FIGS. 1 and 2, the stuffed toy 1 includes a body 5 and a holder 21. The body 5 includes a head 10 and a trunk 20. The holder 21 is located in the trunk 20. The stuffed toy 1 includes a coupling portion 15, serving as a neck. The stuffed toy 1 is configured as a character in such a manner that the head 10 and the trunk 20 are integrated in one piece with the coupling portion 15 located therebetween. The body 5 has an outer surface 5s with a long-pile raised surface texture that is pleasant to touch. The holder 21 includes a hollow space to hold the heat storage member 30.

The stuffed toy 1 can be played such that the head 10 is turned (at the coupling portion 15) relative to the trunk 20 of the character. As will be described later, the stuffed toy 1 is configured such that the head 10 is movable in various ways.

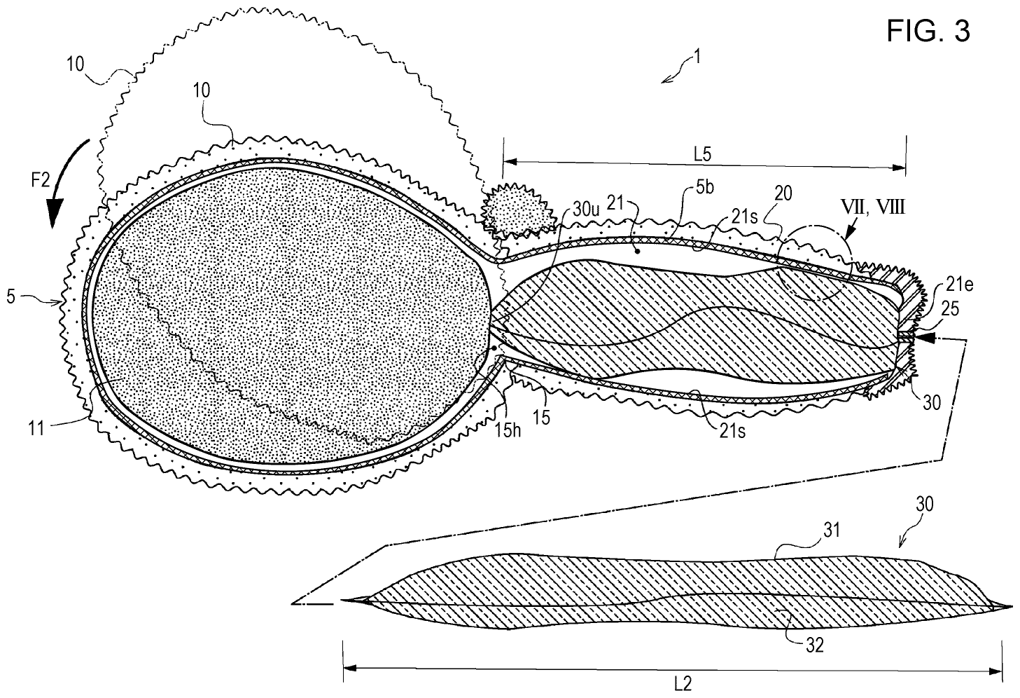

The heat storage member 30 is, for example, a flexible ice pack for cooling, and includes a flexible bag 31 made of synthetic resin and a substance 32 enclosed in the bag 31 and containing, for example, a superabsorbent polymer and water. The heat storage member 30 is rectangular in plan view and has a predetermined size with a lateral dimension L1 and a longitudinal dimension L2. The holder 21 in the trunk 20 also has a rectangular shape. The holder 21 has a lateral dimension L4 and a longitudinal dimension L5, which are less than the lateral and longitudinal dimensions L1 and L2 of the heat storage member 30, respectively. The body 5 includes a stretchy fabric 5b (refer to FIG. 3), serving as a base material. This enables the heat storage member 30, which is larger than the holder 21, to be retained and held tightly.

The holder 21 has an open end 21e having an opening dimension L3, which is less than the lateral dimension L4 of the holder 21. This enables the heat storage member 30 held in the holder 21 to be retained without falling out of the holder 21 even when the open end 21e is left open. The open end 21e has a closure 25, serving as a fastening mechanism such as a hook-and-loop fastener, extending partly or wholly through the open end 21e.

The body 5 includes a retainer 50, such as a strap, located at any position on the head 10. Therefore, the stuffed toy 1 can be attached to another object via the retainer 50 and be enjoyed.

As illustrated in FIG. 2, the head 10 of the body 5 has a relatively round shape with a first thickness T1 in side view. The trunk 20 has a second thickness T2 less than the first thickness T1 of the head 10. The head 10 includes an elastic member 11 (refer to FIG. 3) having appropriate cushioning properties and made of, for example, cotton. Unlike the trunk 20, the head 10 includes no storage space for the heat storage member 30.

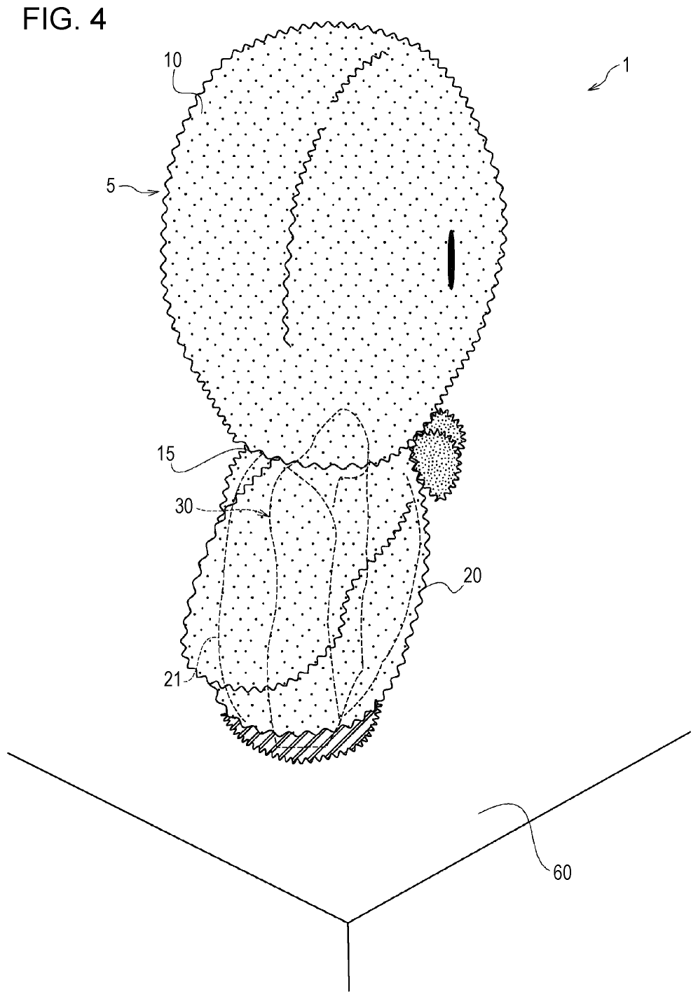

FIG. 3 is a sectional view of the stuffed toy 1 taken along line III-III in FIG. 1 illustrating a side elevation of the heat storage member 30 that is to be held and a side elevation of the heat storage member 30 in a first holding state. FIG. 4 is a perspective view of the stuffed toy 1 in a first display state.

In the body 5, as illustrated in FIG. 3, an inner portion of the head 10 communicates with an inner portion of the trunk 20 via a communication opening 15h at the coupling portion 15. The head 10 is filled with the elastic member 11. Therefore, the head 10 has a rounded appearance having a substantially spherical shape. The holder 21 is configured to hold the heat storage member 30. In the first holding state in which the heat storage member 30 is held as it is, the thickness of the trunk 20 is less than that of the head 10 in side view.

In the holding state illustrated in FIG. 3, the open end 21e is closed by the closure 25 such that the heat storage member 30 is slightly pressed into the holder 21 from the open end 21e. This holding state causes the heat storage member 30, which has the longitudinal dimension L2 in a flat state before being placed into the holder 21, to be compressed to the size of the holder 21 having the longitudinal dimension L5. Thus, the heat storage member 30 is held such that the stuffed toy 1 has a slightly increased front-to-rear dimension (extending vertically in FIG. 3).

Furthermore, the holding state in FIG. 3 allows an upper end 30u of the heat storage member 30 to be partly in contact with the elastic member 11. In this state, the unity of the head 10 and the trunk 20, or the degree of tension of the fabric 5b at the coupling portion 15, is higher than that in a state in which the heat storage member 30 is not held (refer to FIG. 2). Therefore, for example, when the head 10 turned frontward (upward in FIG. 3 as indicated by an alternate long and two short dashes line) is released, the head 10 exhibits resilience F2, which allows the head 10 to return to its original position as illustrated in FIG. 3, greater than resilience F1 illustrated in FIG. 2.

After the heat storage member 30 is held in the above-described manner, the trunk 20 and the head 10 of the stuffed toy 1 are structurally coupled to each other. Thus, the trunk 20 serves as a base supporting the head 10. This allows the stuffed toy 1 to stand alone and be displayed on a stand 60 as illustrated in FIG. 4, for example. The position of the heat storage member 30 in the holder 21 is not limited to that in the example illustrated in FIG. 3. The orientation and position of the heat storage member 30 may be changed freely.

In the body 5 with the heat storage member 30 held in the holder 21, the head 10 has elasticity provided by the elastic member 11, and the trunk 20 has elasticity based on the heat storage member 30. The elasticity of the head 10 is significantly different from that of the trunk 20. For example, the trunk 20 exhibits less resilience to surface deformation than the head 10. This provides different feels to a user when the user presses or touches the stuffed toy 1. The user can enjoy the different feels.

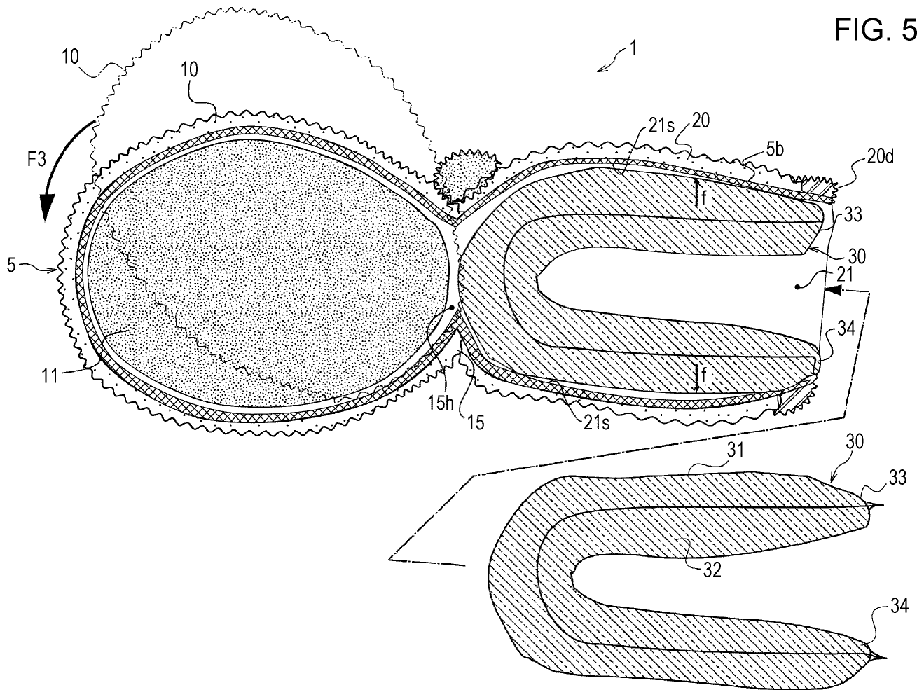

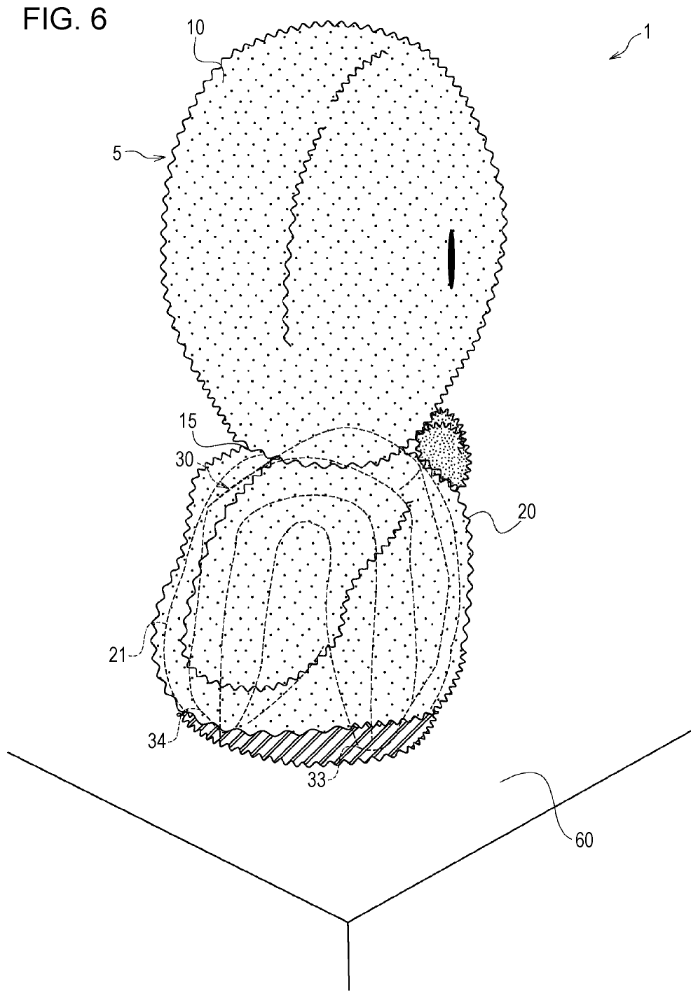

FIG. 5 is a sectional view of the stuffed toy 1 taken along line V-V in FIG. 1 illustrating a side elevation of the heat storage member 30 held in the holder 21 in a second holding state. FIG. 6 is a perspective view of the stuffed toy 1 in a second display state.

FIG. 5 illustrates the state in which the heat storage member 30 is placed in the holder 21. In this state, which differs from that illustrated in FIG. 3, the heat storage member 30 is bent and held in the holder 21. Specifically, the heat storage member 30 is bent at substantially the middle thereof such that opposite ends 33 and 34 of the bent heat storage member 30 are substantially aligned with each other. For example, if the bag 31 contains a relatively large amount of substance 32, a force f may be exerted on the bent heat storage member 30 to increase the angle of bending the heat storage member 30, or extend the bent heat storage member. In this case, such expansion force f can press an inner surface of the trunk 20.

For the thickness of the trunk 20 in side view, which differs from a thin thickness in the first holding state, the trunk 20 can be expanded to substantially the same thickness as that of the head 10. In the state illustrated in FIG. 5, the open end 21e is left open. This allows the trunk 20 to expand, so that the front-to-rear dimension (extending vertically in FIG. 5) of the stuffed toy 1 can be greater than that in FIG. 3.

In the second holding state in FIG. 5, the expansion force f exerted on the heat storage member 30 and pressing the inner surface of the trunk 20 causes the fabric 5b of the trunk 20 to be stretched tighter than in the state illustrated in FIG. 3. Therefore, for example, when the head 10 turned frontward (upward in FIG. 5 as indicated by an alternate long and two short dashes line) is released, the head 10 exhibits resilience F3, which allows the head 10 to return to its original position as illustrated in FIG. 5, greater than the resilience F2 in FIG. 3. Furthermore, a lower end 20d of the trunk 20 is not closed by the closure 25 and is left wide open. The lower end 20d is larger than that in FIG. 3. This allows the stuffed toy 1 to stand alone more stably and be displayed on the stand 60, as illustrated in FIG. 6. In such a placement state, the opposite ends 33 and 34 of the heat storage member 30 exhibit the function of supporting.

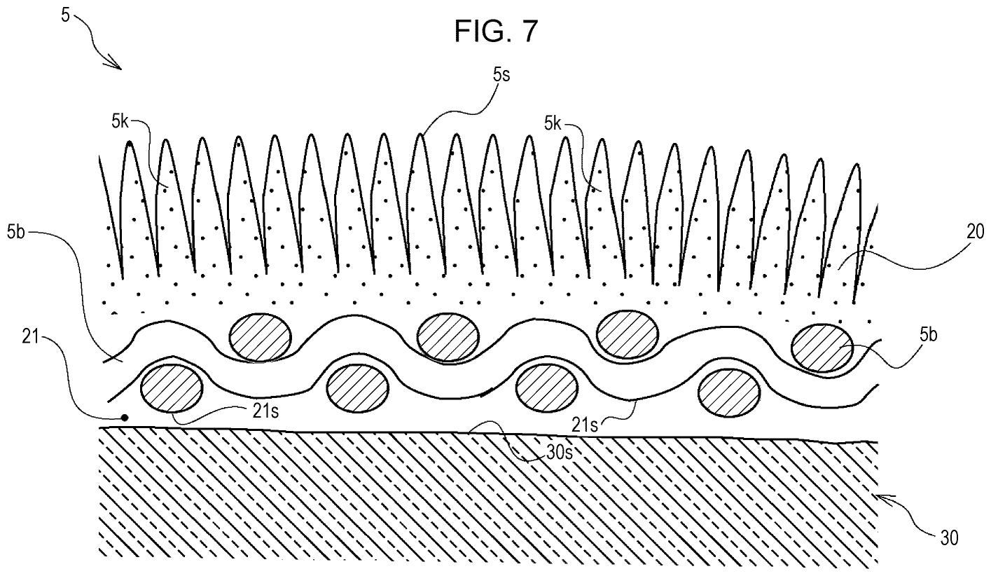

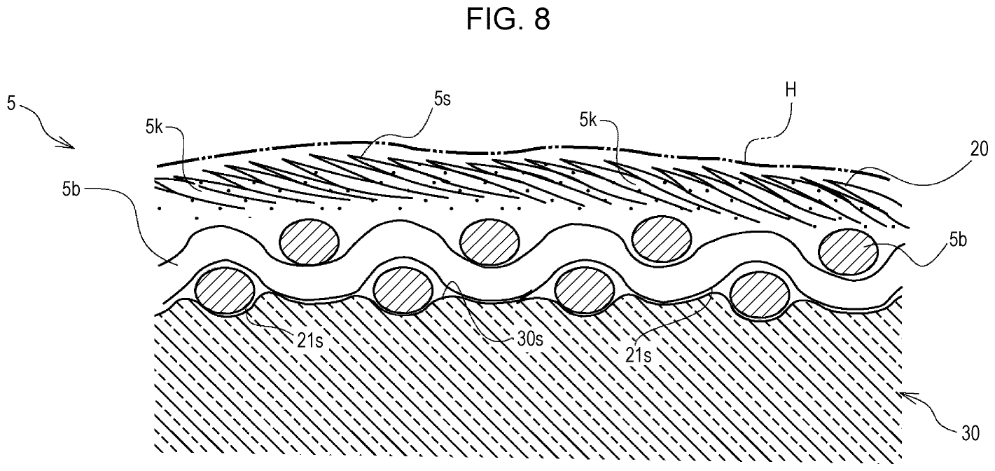

FIG. 7 is an enlarged sectional view of part VII in FIG. 3. FIG. 8 is an enlarged sectional view of part VIII in FIG. 3 subjected to an external force.

The body 5 includes the fabric 5b as described above. As illustrated in the enlarged view of FIG. 7, the stretchy fabric 5b having a woven mesh structure retains a raised surface material 5k included in the outer surface 5s. The holder 21 has an inner surface 21s. The inner surface 21s includes a non-raised surface material having a mesh structure that defines an irregular or uneven surface, as illustrated in FIG. 7. Therefore, the inner surface 21s has irregularities facing an outer surface 30s of the heat storage member 30. Thus, the inner surface 21s is in point contact with the outer surface 30s in enlarged view. Such a point contact state makes it relatively difficult to transfer heat from the heat storage member 30 through the fabric 5b. Since the outer surface 5s includes the raised surface material 5k, the raised surface material 5k traps air therein and thus fluffs. This makes it difficult to transfer heat from the heat storage member 30 to the outside of the stuffed toy 1.

For example, when the trunk 20 is grasped by a hand, the trunk 20 and the heat storage member 30 are deformed to fit the shape of the hand. At this time, as illustrated in FIG. 8, the raised surface material 5k of the outer surface 5s are pressed and compressed between the fabric 5b and the hand, indicated by H. In addition, the inner surface 21s of the holder 21 is in tight contact with the outer surface 30s of the heat storage member 30. In other words, the outer surface 30s of the heat storage member 30 is deformed to fit the uneven surface of the fabric 5b. Thus, the outer surface 30s and the inner surface 21s are in contact with each other with a large area of contact therebetween, as different from those in the state illustrated in FIG. 7. This state with such a large contact of area facilitates heat transfer from the heat storage member 30 through the fabric 5b. Furthermore, the compressed state of the raised surface material 5k of the outer surface 5s causes heat from the heat storage member 30 to be efficiently transferred to the hand H.

As described above, the stuffed toy 1 according to the embodiment is configured such that the holder 21 in the trunk 20 is capable of holding the heat storage member 30. Such a configuration allows the user to enjoy a change in appearance between a holding state of the stuffed toy 1 with the heat storage member 30 held and a non-holding state of the stuffed toy 1 with no heat storage member 30. In addition, the user can enjoy a change in feel, such as touch, (cold or warm to the touch) due to heat from the heat storage member 30 between the holding state with the heat storage member 30 held and the non-holding state with no heat storage member 30.

In the stuffed toy 1 according to the embodiment, the trunk 20 holding the heat storage member 30 differs in elasticity and touch from the head 10 holding, for example, cotton. The head 10 of the stuffed toy 1 differs from the trunk 20 not only in thickness but also in touch. These differences (in, for example, shape, hardness, and thermal sensation upon contact) can be represented as features of a character, thus allowing enjoyment of the stuffed toy 1.

In the stuffed toy 1 according to the embodiment, the head 10 holds the elastic member 11, whereas the trunk 20 can be in either of the two states, or the state with the heat storage member 30 held and a hollow state with no heat storage member 30. This allows the user to enjoy a change in state of the character. Additionally, since the head 10 including a face holds the elastic member 11, such as cotton, facial features of the character can be always accurately represented with a predetermined thickness while remaining unchanged.

In the stuffed toy 1 according to the embodiment, the trunk 20 includes a stretchy material. This allows the holder 21 to change in size. Thus, the holder 21 is capable of holding any of different sized heat storage members 30, thereby allowing the trunk 20 to significantly change in size or shape.

In the stuffed toy 1 according to the embodiment, the heat storage member 30 can be retained in the holder 21 even while the open end 21e of the holder 21 is left open. As long as the open end 21e is left open and located at the bottom while the heat storage member 30 is being held, the trunk 20, serving as a base, facilitates display of the stuffed toy 1 standing alone.

In the stuffed toy 1 according to the embodiment, the position or orientation of the heat storage member 30 in the holder 21 can be changed freely. This allows the user not only to enjoy a change in shape of the trunk 20 but also to easily grasp the trunk 20 with a change in position of the heat storage member 30, for example, when holding the trunk 20 with their hand.

The stuffed toy 1 according to the embodiment is bendable at the coupling portion 15 coupling the head 10 to the trunk 20. Such a configuration allows the user to turn the head 10 relative to the trunk 20 and play with the stuffed toy 1. In addition, this configuration enables the resilience, which allows the head 10 to return to its original or unbent state, of the head 10 turned in the holding state with the heat storage member 30 held in the holder 21 to differ from the resilience of the head 10 turned in the non-holding state. This allows the user to enjoy a change in motion between the holding state and the non-holding state.

The stuffed toy 1 according to the embodiment is configured such that the trunk 20 and the heat storage member 30 are deformable when the user grasps the trunk 20 with the heat storage member 30 held in the holder 21. Such a configuration allows the heat storage member 30 to fit a hand or another part of the user such that the stuffed toy 1 is substantially in tight contact with the hand or the other part.

In the stuffed toy 1 according to the embodiment, the outer surface 5s of the trunk 20 includes the raised surface material. Such a configuration enhances heat retention in the heat storage member 30 inside the holder 21, thus allowing the heat storage member 30 to be maintained at desired temperature. In contrast, the inner surface 21s of the holder 21 includes the non-raised surface material. This configuration allows the inner surface 21s to be in surface contact with the heat storage member 30 held in the holder 21.

In addition, the inner surface 21s of the holder 21 is configured as an uneven surface. This allows the outer surface 30s of the heat storage member 30 to fit the uneven surface and be brought into surface contact with the inner surface 21s under pressure applied to the trunk 20, for example, when the trunk 20 is grasped. This increases the area of contact between the inner surface 21s of the holder 21 and the outer surface 30s of the heat storage member 30, thus improving heat dissipation toward the outer surface 5s of the trunk 20. While the trunk 20 is not pressed, the uneven surface, serving as the inner surface 21s, allows the outer surface 30s of the heat storage member 30 to be substantially in point contact with the inner surface 21s of the holder 21. This leads to improved heat retention in the holder 21.

The stuffed toy 1 according to the embodiment is configured such that the inner surface 21s of the holder 21 has irregularities defined by a mesh structure surface. Such a configuration allows the holder 21 to have a woven warp and weft structure of fibers. This enables the holder 21 to be stretchy.

The stuffed toy 1 according to the embodiment is configured such that the holder 21 of the trunk 20 has a rectangular shape in front view. Such a configuration allows the heat storage member 30 having a rectangular shape to be easily placed in the holder 21. In addition, the holder 21 is capable of holding the heat storage member 30 in a bent state. As long as the opposite ends 33 and 34 of the heat storage member 30 in the bent state are located at the lower end of the trunk 20 (such that a bent part of the heat storage member 30 is located above the lower end), the opposite ends 33 and 34 are apart from each other. This facilitates placement of the stuffed toy 1.

In the stuffed toy 1 according to the embodiment, the inner portion of the head 10 can communicate with the inner portion of the trunk 20 via the communication opening 15h inside the coupling portion 15. This allows the heat storage member 30 held in the holder 21 to extend into the head 10 through the communication opening 15h. This can change the strength of the coupling portion 15. In addition, this enables the head 10 to move in response to, for example, a change in grasping force (or a change in grip strength) exerted on the trunk 20 while the trunk 20 is being grasped.

In the stuffed toy 1 according to the embodiment, the head 10 of the body 5 has the retainer 50 that is attachable to another object. Thus, the stuffed toy 1 can be hung with the head 10 on an upper side.

Although the embodiment of the present invention has been described above, the present invention can be appropriately changed within the scope of technical idea of the present invention. For example, the heat storage member 30 in the above-described embodiment is an ice pack. The heat storage member 30 may include a flexible heat pack.

In the above-described embodiment, the head 10 and the trunk 20 are formed with the same fabric 5b. The configuration is not limited to this example. At least the trunk 20 may include a stretchy material.

In the above-described embodiment, the inner surface 21s of the holder 21 is the uneven surface defined by the mesh structure of the fabric 5b. The structure of the uneven surface is not limited to this example. For example, the fabric 5b may have irregularities.

As described above, the following items are discussed herein.

(1)

A stuffed toy including:
a body including a head and a trunk; and
a holder located in the trunk,
the holder being configured to hold a heat storage member.

(2)

The stuffed toy according to (1), wherein the head excludes the holder for the heat storage member.

(3)

The stuffed toy according to (1) or (2), wherein the head and the trunk are integrated in one piece to represent a character.

(4)

The stuffed toy according to any one of (1) to (3), wherein
the head has a first thickness, and
the trunk has a second thickness less than the first thickness.

(5)

The stuffed toy according to any one of (1) to (4), wherein
the head includes an elastic member, and
the trunk includes a hollow space.

(6)

The stuffed toy according to (5), wherein the elastic member includes cotton.

(7)

The stuffed toy according to any one of (1) to (6), wherein the trunk includes a stretchy material.

(8)

The stuffed toy according to any one of (1) to (7), wherein the head and the trunk exhibit different elasticities in the body with the heat storage member held in the holder.

(9)

The stuffed toy according to any one of (1) to (8), wherein the trunk exhibits less resilience to surface deformation than the head in the body with the heat storage member held in the holder.

(10)

The stuffed toy according to any one of (1) to (9), wherein the holder allows the heat storage member held in the holder to change in position.

(11)

The stuffed toy according to any one of (1) to (10), wherein the body includes a coupling portion coupling the head to the trunk and is bendable at the coupling portion.

(12)

The stuffed toy according to (11), wherein the body with the heat storage member held in the holder and bent at the coupling portion is capable of returning to an unbent state.

(13)

The stuffed toy according to (12), wherein the body with the heat storage member held in the holder and bent at the coupling portion exhibits greater resilience than the body with no heat storage member in the holder and bent at the coupling portion.

(14)

The stuffed toy according to any one of (1) to (13), wherein the trunk and the heat storage member are deformable in the body with the heat storage member held in the holder when the trunk is grasped.

(15)

The stuffed toy according to any one of (1) to (14), wherein the heat storage member held in the holder serves as a base for the body while the body is standing alone.

(16)

The stuffed toy according to any one of (1) to (15), wherein the trunk includes an open end through which the heat storage member is insertable into the holder.

(17)

The stuffed toy according to (16), wherein the trunk includes a closure configured to close the open end, and

the body with no heat storage member in the holder and with the closure closed is incapable of standing alone.

(18)

The stuffed toy according to any one of (1) to (17), wherein as long as the heat storage member is held in the holder, the trunk is capable of standing alone.

(19)

The stuffed toy according to any one of (1) to (18), wherein

the trunk has an outer surface including a raised surface material, and

the holder has an inner surface including a non-raised surface material.

(20)

The stuffed toy according to any one of (1) to (19), wherein the trunk transfers heat more easily than the head.

(21)

The stuffed toy according to (19) or (20), wherein the inner surface of the holder is configured as an uneven surface.

(22)

The stuffed toy according to (21), wherein the uneven surface is a mesh structure surface.

(23)

The stuffed toy according to (1) to (22), wherein the holder is configured to hold the heat storage member in a bent state.

(24)

The stuffed toy according to any one of (1) to (23), wherein the trunk has a rectangular shape in front view.

(25)

The stuffed toy according to any one of (1) to (24), wherein the head includes an inner portion communicating with an inner portion of the trunk.

(26)

The stuffed toy according to any one of (1) to (25), wherein the body includes a retainer located on the head, the retainer being configured to be attached to another object.

What is claimed is:

1. A stuffed toy comprising:
a body including a head and a trunk; and
a holder located in the trunk,
the holder being configured to hold a heat storage member.

2. The stuffed toy according to claim 1, wherein the head excludes the holder for the heat storage member.

3. The stuffed toy according to claim 2, wherein the head and the trunk are integrated in one piece to represent a character.

4. The stuffed toy according to claim 3, wherein the head has a first thickness, and
the trunk has a second thickness less than the first thickness.

5. The stuffed toy according to claim 4, wherein
the head includes an elastic member, and
the trunk includes a hollow space.

6. The stuffed toy according to claim 5, wherein the elastic member comprises cotton.

7. The stuffed toy according to claim 6, wherein the trunk comprises a stretchy material.

8. The stuffed toy according to claim 7, wherein the head and the trunk exhibit different elasticities in the body with the heat storage member held in the holder.

9. The stuffed toy according to claim 8, wherein the trunk exhibits less resilience to surface deformation than the head in the body with the heat storage member held in the holder.

10. The stuffed toy according to claim 9, wherein the holder allows the heat storage member held in the holder to change in position.

11. The stuffed toy according to claim 1, wherein the body includes a coupling portion coupling the head to the trunk and is bendable at the coupling portion.

12. The stuffed toy according to claim 11, wherein the body with the heat storage member held in the holder and bent at the coupling portion is capable of returning to an unbent state.

13. The stuffed toy according to claim 12, wherein the body with the heat storage member held in the holder and bent at the coupling portion exhibits greater resilience than the body with no heat storage member in the holder and bent at the coupling portion.

14. The stuffed toy according to claim 1, wherein the trunk and the heat storage member are deformable in the body with the heat storage member held in the holder when the trunk is grasped.

15. The stuffed toy according to claim 1, wherein the heat storage member held in the holder serves as a base for the body while the body is standing alone.

16. The stuffed toy according to claim 1, wherein the trunk includes an open end through which the heat storage member is insertable into the holder.

17. The stuffed toy according to claim 16, wherein
the trunk includes a closure configured to close the open end, and
the body with no heat storage member in the holder and with the closure closed is incapable of standing alone.

18. The stuffed toy according to claim 17, wherein as long as the heat storage member is held in the holder, the trunk is capable of standing alone.

19. The stuffed toy according to claim 18, wherein
the trunk has an outer surface comprising a raised surface material, and
the holder has an inner surface comprising a non-raised surface material.

20. The stuffed toy according to claim 19, wherein the trunk transfers heat more easily than the head.

21. The stuffed toy according to claim 20, wherein the inner surface of the holder is configured as an uneven surface.

22. The stuffed toy according to claim 21, wherein the uneven surface is a mesh structure surface.

23. The stuffed toy according to claim 22, wherein the holder is configured to hold the heat storage member in a bent state.

24. The stuffed toy according to claim 23, wherein the trunk has a rectangular shape in front view.

25. The stuffed toy according to claim 24, wherein the head includes an inner portion communicating with an inner portion of the trunk.

26. The stuffed toy according to claim 25, wherein the body includes a retainer located on the head, the retainer being configured to be attached to another object.
